(19) United States
(12) Patent Application Publication
(10) Pub. No.: US 2022/0249523 A1
(43) Pub. Date: Aug. 11, 2022

(54) BENZOAZEPINE COMPOUND-CONTAINING FREEZE-DRIED COMPOSITION

(71) Applicant: OTSUKA PHARMACEUTICAL CO., LTD., Tokyo (JP)

(72) Inventors: Takakuni MATSUDA, Osaka-shi, Osaka (JP); Nobutomo SAKO, Osaka-shi, Osaka (JP)

(73) Assignee: OTSUKA PHARMACEUTICAL CO., LTD., Tokyo (JP)

(21) Appl. No.: 17/435,905

(22) PCT Filed: Mar. 27, 2020
(86) PCT No.: PCT/JP2020/013924

§ 371 Date: Sep. 2, 2021

(30) Foreign Application Priority Data
Mar. 28, 2019 (JP) 2019-064347

(51) Int. Cl. A61K 31/675; A61K 9/19; A61K 9/16; A61K 9/08

(52) U.S. Cl. CPC A61K 31/675; A61K 9/08; A61K 9/1623; A61K 9/19

(57) ABSTRACT

A formulation stably comprising a tolvaptan prodrug is provided. Specifically, a freeze-dried composition comprising a compound represented by Formula (1):

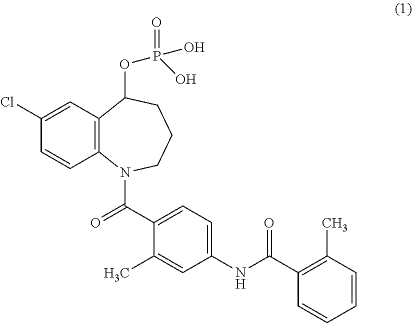 [SMILES: Cc1ccccc1C(=O)Nc1ccc(C(=O)N2CCCC(OP(=O)(O)O)c3cc(Cl)ccc32)c(C)c1]

or a metal salt thereof and a disaccharide is provided.

### TECHNICAL FIELD

The present disclosure relates to a benzoazepine compound-containing freeze-dried composition. All of the documents disclosed herein, including the following prior art documents (Patent Literature (PTL) and Non-Patent Literature (NPL)), are incorporated herein by reference in their entirety.

### BACKGROUND ART

Tolvaptan, which is a benzoazepine compound, has vasopressin V2 receptor antagonistic activity, and is used as a diuretic etc. The following Formula (2) shows the structural formula of tolvaptan.

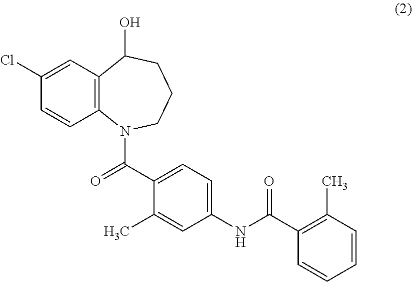 [SMILES: Cc1ccccc1C(=O)Nc1ccc(C(=O)N2CCCC(O)c3cc(Cl)ccc32)c(C)c1]

However, tolvaptan is poorly water-soluble, and there are many restrictions in terms of dosage form, administration route, and the like. Tolvaptan can be administered to patients for whom oral administration is difficult (patients with swallowing difficulties, or patients who are unconscious), and the effect of the medicament is anticipated to be expressed more rapidly than tablets; thus, an injection formulation of tolvaptan for transvascular administration was demanded. However, the development of tolvaptan, which has poor water solubility, was difficult. Thus, research and development have been conducted on a tolvaptan prodrug that is water-soluble. For example, PTL 1 proposes a tolvaptan prodrug having excellent water solubility.

### CITATION LIST

### Patent Literature

### [PTL 1]

### WO2007/074915

### SUMMARY OF INVENTION

### Technical Problem

However, a prodrug of tolvaptan is poorly stable, and is easily converted back into tolvaptan. Therefore, the present inventors have attempted to develop a formulation that stably comprises a tolvaptan prodrug.

### Solution to Problem

The present inventors analyzed the stability of a tolvaptan prodrug. They noted that in a process for preparing an aqueous solution formulation, such as an injection, the prodrug, when dissolved in water and sterilized with high-pressure steam, was degraded to form poorly water-soluble tolvaptan, resulting in cloudiness or formation of insoluble particulate matter. Further, long-term storage of the aqueous solution formulation caused degradation of the prodrug to form tolvaptan, resulting in precipitation of insoluble foreign matter or formation of insoluble particulate matter.

In view of the above, the present inventors conducted further research on the stability of the tolvaptan prodrug, and found that a composition comprising a specific tolvaptan prodrug and a disaccharide can possibly comprise the prodrug stably. The inventors then made further improvements.

For example, the present disclosure encompasses the subject matter presented in the following items.

Item 1. A freeze-dried composition comprising a compound represented by Formula (1):

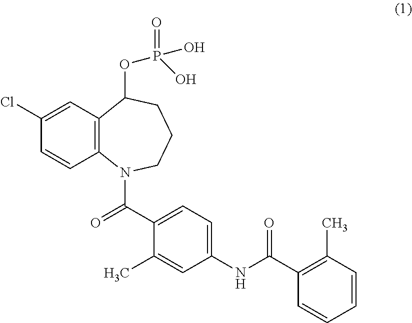 [SMILES: Cc1ccccc1C(=O)Nc1ccc(C(=O)N2CCCC(OP(=O)(O)O)c3cc(Cl)ccc32)c(C)c1]

or a metal salt thereof and a disaccharide.

Item 2. The freeze-dried composition according to claim 1, wherein the metal salt is a disodium salt.

Item 3. The freeze-dried composition according to Item 1 or 2, wherein the disaccharide is at least one member selected from the group consisting of sucrose, maltose, lactose, and trehalose.

Item 4. The freeze-dried composition according to any one of claims 1 to 3, wherein the disaccharide is present in an amount of 0.5 to 70 parts by mass per part by mass of the compound represented by Formula (1) or a metal salt thereof.

Item 5. The freeze-dried composition according to any one of Items 1 to 4, wherein the total amount of the compound represented by Formula (1) or a metal salt thereof and the disaccharide is 65% by mass or more of the entire composition.

Item 6. The freeze-dried composition according to any one of items 1 to 5, further comprising a buffering agent.

Item 7. The freeze-dried composition according to Item 6, wherein the buffering agent is a phosphate buffering agent.

Item 8. The freeze-dried composition according to any one of Items 1 to 7, which is for use by transvascular administration after dissolving in water (preferably a physiological saline or a glucose injection solution) so as to constitute an aqueous solution composition having a pH of 7 to 9.

Item 9. An aqueous solution composition comprising a compound represented by Formula (1):

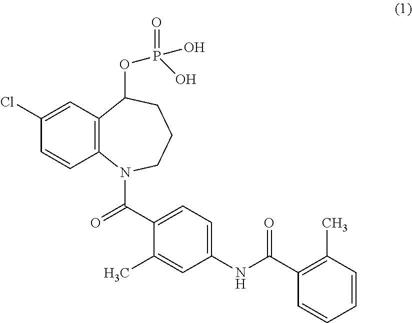 [SMILES: Cc1ccccc1C(=O)Nc1ccc(C(=O)N2CCCC(OP(=O)(O)O)c3cc(Cl)ccc32)c(C)c1]

or a metal salt thereof and a disaccharide, and having a pH of 7 to 9.

Item 10. The aqueous solution composition according to item 9, wherein the metal salt is a disodium salt.

Item 11. The aqueous solution composition according to item 9 or 10, wherein the disaccharide is at least one member selected from the group consisting of sucrose, maltose, lactose, and trehalose.

Item 12, The aqueous solution composition according to any one of Items 9 to 11, wherein the disaccharide is present in an amount of 0.5 to 70 parts by mass per part by mass of the compound represented by Formula (1) or a metal salt thereof.

Item 13. The aqueous solution composition according to any one of items 9 to 12, wherein the disaccharide is present at a concentration of 1 to 8 w/v %.

Item 14. The aqueous solution composition according to any one of Items 9 to 13, further comprising a buffering agent.

Item 15. The aqueous solution composition according to Item 14, wherein the buffering agent is a phosphate buffering agent.

Item 16. The aqueous solution composition according to any one of items 9 to 15, which is for transvascular administration.

Item 17. The aqueous solution composition according to any one of Items 9 to 16, for use in the preparation of the freeze-dried composition of any one of Items 1 to 8.

Item 18. The freeze-dried composition according to any one of Items 1 to 8, for use in the preparation of the aqueous solution composition of any one of items 9 to 16.

Item 19. The composition according to any one of Items 1 to 18, which is sterilized.

Item 20. The composition according to any one of Items 1 to 19, which is a pharmaceutical composition.

### Advantageous Effects of Invention

A formulation (preferably an aqueous injection) that stably comprises a specific tolvaptan prodrug is provided. Since aqueous injections are used for transvascular administration, the formation of insoluble foreign matter or insoluble particulate matter in the solution exceeding the number specified in the Pharmacopoeia of each country is not allowed. The formulation stably comprising a tolvaptan prodrug is particularly suitable as an aqueous injection because the formulation does not easily form insoluble foreign matter or insoluble particulate matter equal to or greater than the number that is specified in the Pharmacopoeia, even after long-term storage.

### BRIEF DESCRIPTION OF DRAWINGS

FIG. 1 is graphs showing the results of evaluation of stability aqueous solutions prepared to contain compound (1) at a concentration of 0.1 w/v %, and subjected to high-pressure steam sterilization (121° C., 20 minutes).

FIG. 2 is a graph showing the results of evaluation of stability of freeze-dried compositions of compound (1) prepared by using aqueous solutions with different pHs.

### DESCRIPTION OF EMBODIMENTS

The present disclosure preferably encompasses, for example, a freeze-dried composition or an aqueous solution composition comprising a specific tolvaptan prodrug; however, the disclosure is not limited to these, and encompasses all of the matter disclosed herein and recognized by a person skilled in the art.

The freeze-dried composition and aqueous solution composition encompassed by the present disclosure each comprise a compound represented by the following Formula (1):

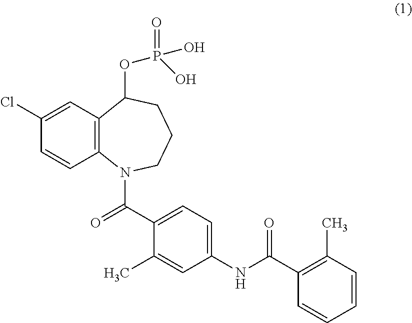 [SMILES: Cc1ccccc1C(=O)Nc1ccc(C(=O)N2CCCC(OP(=O)(O)O)c3cc(Cl)ccc32)c(C)c1]

or a metal salt thereof and a disaccharide, and preferably comprise a metal salt of the compound represented by Formula (1) and a disaccharide. The compound represented by Formula (1) is sometimes referred to as “compound (1).” Further, the freeze-dried composition and the aqueous solution composition each comprising compound (1) or a salt thereof and a disaccharide are sometimes referred to as “the freeze-dried composition according to the present disclosure” and “the aqueous solution composition according to the present disclosure,” respectively. They are sometimes collectively referred to as “the compositions according to the present disclosure.” It is preferable that the freeze-dried composition according to the present disclosure be prepared by freeze-drying the aqueous solution composition according to the present disclosure. It is further preferable that the aqueous solution composition according to the present disclosure be prepared by reconstituting with water the freeze-dried composition according to the present disclosure. The freeze-dried composition according to the present disclosure is preferably a cake-like composition.

Compound (1) or a metal salt thereof serves as a specific tolvaptan prodrug, which is contained in the freeze-dried composition or aqueous solution composition according to the present disclosure. In particular, the specific tolvaptan prodrug is preferably a metal salt of compound (1).

The metal salt of compound (1) is preferably an alkali metal salt, an alkaline earth metal salt, or a zinc salt. More specifically, for example, a sodium salt (mono or disodium salt), a potassium salt (mono or dipotassium salt), a calcium salt, a magnesium salt, a zinc salt, and the like are preferable. Of these, a disodium salt is particularly preferable. The following is the structural formula of a disodium salt of compound (1).

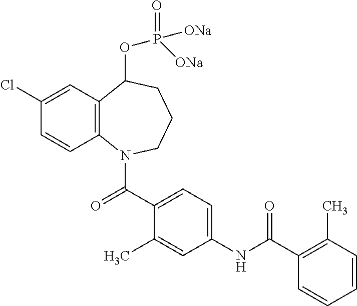

Compound (1) or a metal salt thereof can be produced by a known method, or a method easily conceivable of from a known method. For example, they can be produced by the method disclosed in PTL 1 (WO2007/074915) (in particular, the method disclosed in the Examples).

The disaccharide is preferably a disaccharide in which at least one of the two saccharides constituting the disaccharide is glucose. Specific examples include sucrose, maltose, trehalose, lactose, cellobiose, and the like, with sucrose, maltose, trehalose, and lactose being preferable, sucrose and trehalose being more preferable, and sucrose being particularly preferable. These disaccharides can be used alone, or in a combination of two or more.

The disaccharide is preferably present in an amount of about 0.5 to 70 parts by mass per part by mass of the amount of compound (1) or a metal salt thereof. The upper or lower limit of the range may be, for example, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69 parts by mass. For example, the amount of disaccharide may be about 0.8 to 60 parts by mass per part by mass of the amount of compound (1) or a metal salt thereof. In consideration of foaming in reconstitution of the freeze-dried composition according to the present disclosure with water, the amount is preferably about 1 to 15 parts by mass, since foaming is less likely to occur.

In particular, when the composition is a freeze-dried composition, the total amount of compound (1) or a metal salt thereof and a disaccharide is preferably 65% by mass or more, and more preferably 66, 67, 68, 69, or 70% by mass or more, of the entire composition.

In particular, when the composition is an aqueous solution composition, the disaccharide is preferably present at a concentration of 1 to 8 w/v %. The upper or lower limit of the range may be, for example, 1.5, 2, 2.5, 3, 3.5, 4, 4.5, 5, 5.5, 6, 6.5, 7, or 7.5 w/v %. For example, in consideration of foaming in reconstitution, with water, of the freeze-dried composition according to the present disclosure, the concentration is preferably 1 to 3 w/v %, since foaming is less likely to occur.

The compositions according to the present disclosure preferably further comprise a buffering agent. The buffering agent is preferably a phosphate buffering agent or a carbonate buffering agent, and particularly preferably a phosphate buffering agent. More specifically, for example, disodium hydrogen phosphate (sodium hydrogen phosphate) and/or sodium dihydrogen phosphate are preferable. The concentration of the phosphate buffering agent in the aqueous solution composition is not particularly limited, as long as the buffer capacity is exhibited. The concentration is preferably, for example, about 5 to 100 mM. The upper or lower limit of the range may be, for example, 10, 15, 20, 25, 30, 35, 40, 45, 50, 55, 60, 65, 70, 80, 85, 90, or 95 mM. For example, the concentration is more preferably about 10 to 80 mM, further preferably about 15 to 50 mM, and still more preferably about 20 to 40 mM.

Further, the compositions according to the present disclosure may optionally comprise a pH adjusting agent. In terms of the pH adjusting agents, specific examples of acidic pH adjusting agents include hydrochloric acid, acetic acid, phosphoric acid, and the like; and specific examples of basic pH adjusting agents include sodium hydroxide, potassium hydroxide, calcium carbonate, magnesium oxide, magnesium hydroxide, and the like. Since the aqueous solution composition according to the present disclosure has a pH of 6.5 to 9, it is particularly preferable to use a basic pH adjusting agent. Of these, sodium hydroxide is particularly preferable. The upper or lower limit of the pH range of the aqueous solution composition according to the present disclosure may be, for example, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, or 8.9. For example, the aqueous solution composition according to the present disclosure preferably has a pH of about 8 to 9, and most preferably about 8.5.

In addition to the above, the compositions according to the present disclosure may further optionally comprise a pharmaceutically acceptable carrier, in particular, a component known in the field of freeze-dried pharmaceutical formulations.

The compositions according to the present disclosure can be preferably used as, for example, a pharmaceutical composition. In particular, the compositions can be preferably used as a vasopressin receptor (in particular, V2 receptor) antagonist. More specifically, for example, the pharmaceutical composition can be preferably used to treat congestive heart failure, liver cirrhosis, or hyponatremia due to the syndrome of inappropriate antidiuretic hormone secretion (SIADH); or inhibit an increase in kidney volume or a decrease in kidney function in autosomal dominant polycystic kidney disease.

The administration route of the compositions according to the present disclosure is preferably, but not particularly limited to, transvascular administration, and more preferably intravenous administration. The aqueous solution composition can be directly used for trans vascular administration. The freeze-dried composition can be used for transvascular administration after being dissolved in water (i.e., reconstitution). The water for dissolving the freeze-dried composition may contain other components known in this technical field. For example, the water is more preferably a physiological saline, or a glucose injection solution.

Examples of the dosage form of the compositions according to the present disclosure include, but is not particularly limited to, injections, drip infusions, and the like.

As described above, the aqueous solution composition according to the present disclosure can be used for transvascular administration as is. Additionally, by freeze-drying the aqueous solution composition according to the present disclosure, the freeze-dried composition according to the present disclosure can be preferably prepared. That is, the aqueous solution composition according to the present disclosure is also useful for preparing the freeze-dried composition according to the present disclosure.

The aqueous solution composition according to the present disclosure can be preferably prepared by dissolving the freeze-dried composition according to the present disclosure in water (i.e., reconstitution). More specifically, the freeze-dried composition according to the present disclosure can be prepared from the aqueous solution composition according to the present disclosure, and is also useful for (re-)preparing the aqueous solution composition according to the present disclosure.

For use as an injection, the compositions according to the present disclosure is preferably sterilized or aseptic. The sterilization method is not particularly limited. Preferable examples include a method of performing aseptic filtration after preparing the aqueous solution.

The compositions according to the present disclosure can be prepared based on a known method, for example, a method for preparing a freeze-dried pharmaceutical formulation. More specifically, for example, an aqueous solution composition can be prepared by mixing compound (1) or a metal salt thereof and a disaccharide; and optionally a buffering agent, a pH adjusting agent, and the like, together with water for dissolution. Further, as described above, the freeze-dried composition can be prepared by freeze-drying the aqueous solution composition.

The terms “comprising” and “containing” include “consisting essentially of” and “consisting of.”

The various characteristics (e.g., properties, structures, and functions) described in each of the above embodiments may be combined in any way to specify the subject matter encompassed by the present disclosure.

### EXAMPLES

The subject matter encompassed by the present disclosure is described in more detail below. However, the subject matter is not limited to the following Examples.

### Production of Metal Salts of Compound (1)

Compound (1) and a disodium salt thereof were prepared according to the method disclosed in the Examples (in particular, Examples 1, 3, and 9) of PTL 1 (WO2007/074915). The disodium salt was used as compound A in the following analysis. The preparation was specifically performed as follows. In the description of the following specific preparation method, compound (1b) corresponds to compound (1), and the disodium salt of compound (1b) corresponds to compound A.

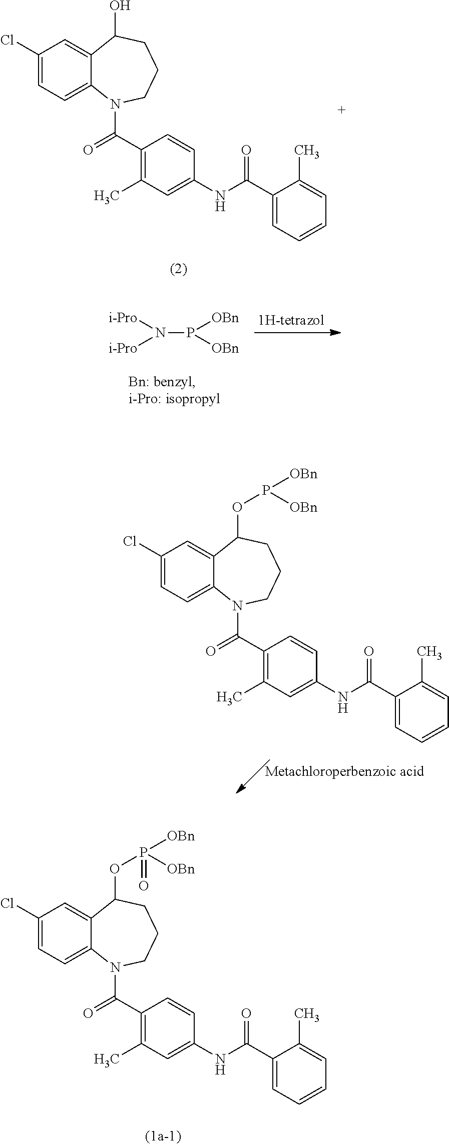

A 1.0 g quantity of tolvaptan and 460 mg of 1H-tetrazole were dissolved in 30 ml of methylene chloride; and 1.2 g of dibenzyl diisopropylphosphoramidite was added dropwise to this solution with stirring at room temperature, followed by stirring at the same temperature for 2 hours.

The obtained reaction mixture was cooled to −40° C., and a solution of 920 mg of metachloroperhenzoic acid in methylene chloride (6 ml) was added dropwise thereto. The mixture was stirred at the same temperature for 30 minutes, and at 0° C. for another 30 minutes. The reaction mixture was washed with an aqueous sodium thiosulfate solution and saturated aqueous sodium bicarbonate solution, and then dried over anhydrous sodium sulfate. The obtained reaction mixture was filtered and concentrated, and the residue was purified by silica gel column chromatography (eluent:n-hexane:ethyl acetate=1:1) to obtain 1.5 g of compound (1a-1) in an amorphous form (yield: 97.2%).

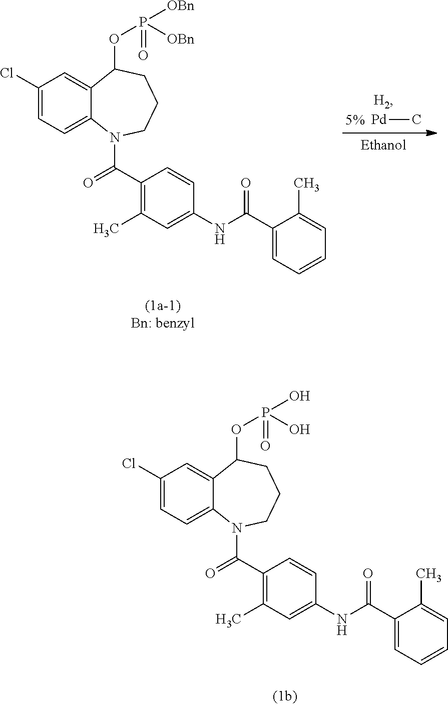

A 5.3 g quantity of compound (1a-1) was dissolved in 100 ml of ethanol. Using 2 g of 5% palladium on carbon as a catalyst, the solution was catalytically reduced at ordinary temperature under ordinary pressure for 10 minutes. The catalyst was removed from the solution by filtration, and the obtained filtrate was concentrated (4.2 g). The obtained residue was crystallized from methanol-water. The crystals were collected by filtration, and dried under reduced pressure (diphosphorus pentoxide) to obtain 3.5 g of compound (1b) as a white powder (yield: 88.5%).

Further, 1.0 ml of 1N aqueous sodium hydroxide solution was added under ice-cooling to a solution of 276 mg (0.52 mmol) of compound (1b) in methanol (2 ml), and the resulting mixture was stirred for 5 minutes. The reaction mixture was concentrated under reduced pressure, and the residue was recrystallized from acetone-water to obtain 221 mg of disodium salt of compound (1b) as a white powder.

Additionally, according to the method disclosed in the Examples of PTL 1, a calcium salt, a magnesium salt, and a zinc salt of compound (1) were produced. The stability of these metal salts in a solid state was analyzed. The disodium salt (i.e., compound A), calcium salt, magnesium salt, and zinc salt had remarkably improved stability, compared to compound (1). Additionally, the solubility in water was analyzed. The disodium salt (i.e., compound A) had remarkably excellent solubility in water, compared to compound (1), calcium salt, magnesium salt, and zinc salt, as shown in the following table; the disodium salt was suitable as a drug substance for aqueous injection formulations.

TABLE 1
Solubility in water of compound (1) and metal salts of
compound (1) (w/v %)

| | Compound | Disodium | Calcium | Magnesium | Zinc |
|---|---|---|---|---|---|
| | (1) | salt | salt | salt | salt |
| Solubility | 0.14 w/v % | 4 w/v % | 0.12 w/v % | 0.14 w/v % | 0.003 |
| | | or more | | | w/v % |

### Stability of Phosphate Ester Bond in Compound (1) in High-Pressure Steam Sterilization Treatment

Aqueous solutions containing compound (1) at a concentration of 0.1 w/v % were prepared, and the stability of the aqueous solutions when subjected to high-pressure steam sterilization (121° C., 20 minutes) was analyzed. The aqueous solutions were prepared using a 100 mM sodium phosphate buffer or a 100 mM Tris buffer. The pH of the aqueous solutions prepared using each of the buffers was adjusted with sodium hydroxide to prepare solutions with different pHs. After the treatment, the purity of compound (1) and the amount of tolvaptan produced were determined by the HPLC area normalization method.

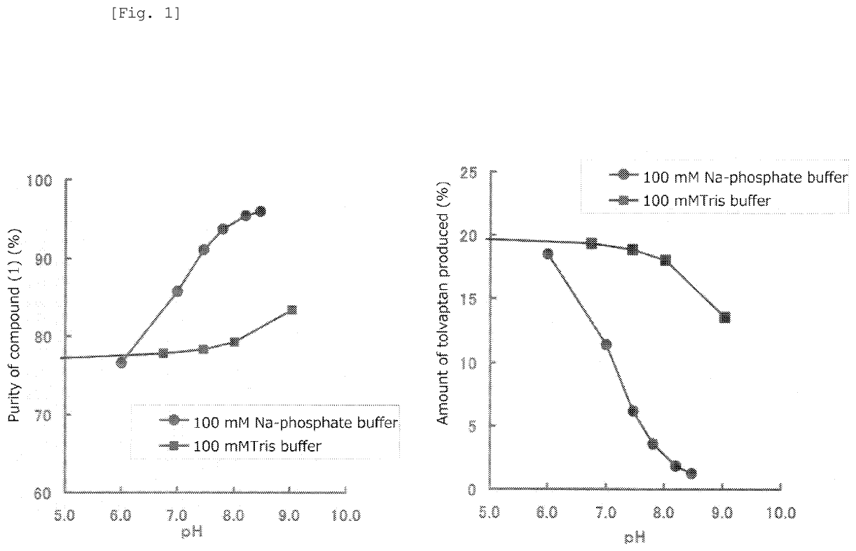

FIG. 1 shows the specific results. The results revealed that the phosphate ester bond in compound (1) was hydrolyzed by high-pressure steam sterilization, and tolvaptan was formed as a precipitate. The results also revealed that the use of a sodium phosphate buffer having a pH of 7.5 or more effectively suppressed the hydrolysis, and the phosphate ester bond in compound (1) was highly stable.

### Analysis 1: Stability of Freeze-Dried Formulation of Compound (1)

Aqueous solutions containing compound (1) (0.1 w/v %), mannitol (4 w/v %), and sodium hydroxide (q.s.) were prepared (pH 7, 7.5, 8, 8.5, or 9) using a 20 mM disodium hydrogen phosphate buffer.

The aqueous solutions were each placed in a 2-mL glass vial, and frozen to a temperature of −40° C. or lower. Thereafter, the vials were depressurized to vacuum, and water was removed during freeze-drying to thus obtain freeze-dried compositions. The resulting products were stored at 40° C. for 3 months, or at 60° C. for 1 month; and then reconstituted with the same amount of water as that removed by freeze-drying, to return the products back to aqueous solutions. The amount of total degradation products or tolvaptan produced (%) in each of the obtained aqueous solutions was determined by the HPLC area normalization method.

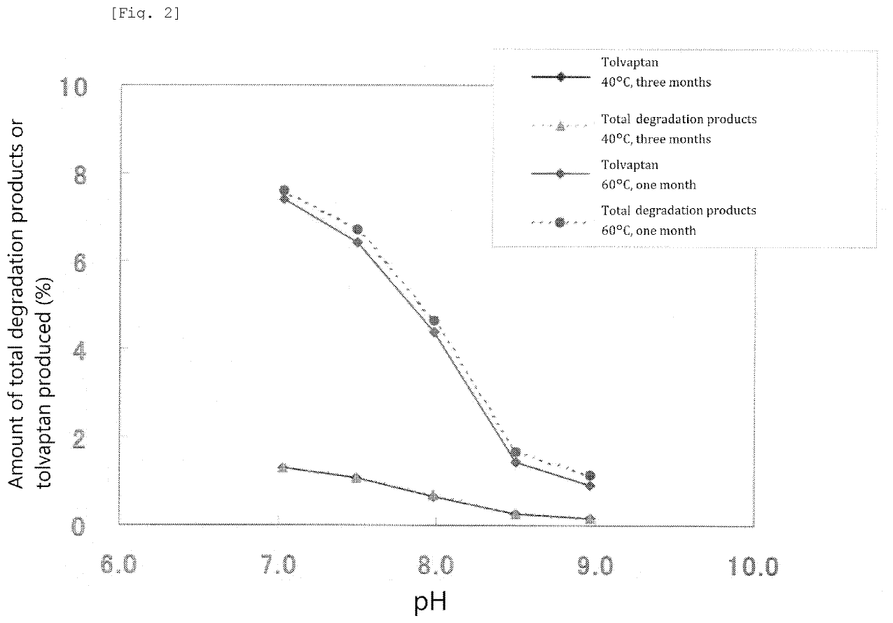

FIG. 2 shows the results. In the freeze-dried compositions prepared using the solutions with a pH exceeding 8, compound (1) was relatively stable, even during storage at 60° C. However, in the freeze-dried compositions that contained mannitol, tolvaptan was formed after storage at 60° C. for 1 month, even though these compositions were prepared using the solutions with a high pH; i.e., 1.45% of tolvaptan was formed when the solution having a pH of 8.5 was used, and 0.9% of tolvaptan was formed when the solution having a pH of 9 was used, thus showing an insufficient stabilization effect.

### Analysis 2: Stability of Freeze-Dried Formulation of Compound (1)

Freeze-dried compositions were prepared in the same manner as in the “Analysis 1: Stability of Freeze-Dried Formulation of Compound (1)” section above using additives other than mannitol, i.e., NaCl, sorbitol, sucrose, maltose, trehalose, or lactose. Here, the pH of the aqueous solutions before preparing freeze-dried compositions was adjusted to 8.5. However, for lactose, the pH of the aqueous solution before preparing a freeze-dried composition was adjusted to 9.0 with a 24 mM sodium hydrogen carbonate buffer.

After the obtained freeze-dried compositions were stored at 60° C. for 1 month, the amount of tolvaptan produced was determined by the HPLC area normalization method, and the stability of compound (1) in each composition was analyzed. Table 2 shows the results. The results confirmed that the addition of a disaccharide (sucrose, maltose, trehalose, or lactose) remarkably reduced the amount of tolvaptan produced, compared to when sodium chloride or mannitol was added. The reduction was particularly remarkable with sucrose, maltose, and lactose. Additionally, after storage at 60° C. for 1 week of the aqueous solutions before preparing freeze-dried compositions, the stability of compound (1) in each composition was analyzed. Table 3 shows the results. The results confirmed that the use of maltose or lactose increased the amount of tolvaptan produced (%).

The above results revealed that the use of a disaccharide for preparing the freeze-dried compositions (i.e., for preparing the aqueous solution compositions for preparing the freeze-dried compositions) remarkably improved the stability of the tolvaptan prodrug (compound (1)) in the freeze-dried compositions, and further that the use of sucrose remarkably improved the stability of the tolvaptan prodrug (compound (1)), even in a state of an aqueous-solution composition that had not been freeze-dried. Accordingly, the results revealed that the addition of sucrose achieved an extremely remarkable stabilization effect in the freeze-dried products and in an aqueous solution state.

TABLE 2
Amount of tolvaptan produced (%) after storage at 60° C. for
1 month of the freeze-dried compositions

| | Amount of tolvaptan produced (%) |
|---|---|
| 0.9% sodium chloride | 5.91 |
| 4% mannitol | 1.45 |
| 4% sucrose | 0.13 |
| 4% maltose | 0.13 |
| 4% trehalose | 0.19 |
| 7.5% lactose (pH 9.0) | Below the detection limit |

TABLE 3
Amount of tolvaptan produced (%) after storage at 60° C. for 1 week
of aqueous solutions before preparing freeze-dried compositions

| | Amount of tolvaptan |
|---|---|
| | produced (%) |
| 4% sucrose | 0.24 |
| 4% maltose | 2.03 |
| 4% trehalose | 0.33 |
| 7.5% lactose (pH 9.0) | 1.12 |

### Analysis 1: Stability of Freeze-Dried Formulation of Compound A

Aqueous solutions with a pH of 7.5, 8, 8.5, or 9 were prepared in the same manner as in the “Analysis 2: Stability of Freeze-Dried Formulation of Compound (1)” section above, except that compound A at a concentration of 0.541 w/v % and sucrose at a concentration of 7.5 w/v % were used. The resulting aqueous solutions were freeze-dried to obtain freeze-dried compositions. The freeze-dried compositions were stored at 60° C. for 1 month. Thereafter, the amount of tolvaptan produced (%) was determined by the HPLC area normalization method. The amount of tolvaptan produced was 0.22% when the pH was 7.5, 0.10% when the pH was 8, 0.07% when the pH was 8.5, and below the detection limit when the pH was 9; all of these showed excellent stability.

### Analysis 2: Stability of Freeze-Dried Formulation of Compound A

An aqueous solution with a pH of 8.5 was prepared in the same manner as in the “Analysis 1: Stability of Freeze-Dried Formulation of Compound A” section above, using potassium phosphate instead of sodium phosphate as a buffering agent. Further, a freeze-dried composition was prepared to analyze the stability of compound A in the freeze-dried composition (after storage at 60° C. for 1 month). The amount of tolvaptan produced (%) was 0.07%, which was the same as when sodium phosphate was used.

Aqueous solutions with a pH of 8.0 were prepared in the same manner as in the “Analysis 1: Stability of Freeze-Dried Formulation of Compound A” section above, such that the sucrose concentration was 1%, 2%, 4%, or 7.5%. Further, the freeze-dried compositions were prepared to analyze the stability of compound A in the freeze-dried compositions (after storage at 60° C. for 1 month). The amount of tolvaptan produced (%) was 0.25% when the sucrose concentration was 1%, 0.12% when the sucrose concentration was 2%, 0.08% when the sucrose concentration was 4%, and 0.10% when the sucrose concentration was 7.5%. Thus, the stability of compound A analyzed here was excellent in all instances; and these results revealed that the aqueous solutions containing sucrose at a concentration, in particular, exceeding 1% exhibited more excellent stability. However, when the freeze-dried compositions with a sucrose concentration of 4% or 7.5% were reconstituted with water and returned to the aqueous solutions, foaming and slight cloudiness were observed; it was necessary for the solutions to be allowed to stand until degassing was completed, and the color returned to transparent. Although this does not cause a problem in administration, it is considered preferable that the sucrose concentration in the aqueous solution be less than 4%, from the viewpoint of having no cloudiness caused by foaming.

In addition to the aqueous solution containing sodium phosphate at a concentration of 20 mM and having a pH of 8.5, aqueous solutions containing sodium phosphate at a concentration of 10 mM or 50 mM were also prepared as in the same manner as the “Analysis 1: Stability of Freeze-Dried Formulation of Compound A” section above. Further, freeze-dried compositions were prepared to analyze the stability of compound A in the freeze-dried compositions (after storage at 60° C. for 1 month, or at 40° C. for 3 months). Thereafter, the pH at the time of reconstitution with water was analyzed. The following table shows the results.

TABLE 4

| | pH at 60° C. | pH at 40° C. |
|---|---|---|
| Sodium phosphate | 1 month later | 3 months later |
| 10 mM | 8.24 | 8.16 |
| 20 mM | 8.34 | 8.24 |
| 50 mM | 8.36 | 8.30 |

The results revealed that no significant change in pH was observed, compared to before the storage.

Further, the stability of compound A in the freeze-dried compositions was analyzed (after storage at 60° C. for 1 month). The amount of tolvaptan produced (%) was 0.07% when the concentration was 10 mM, 0.07% when the concentration was 20 mM, and below the detection limit when the concentration was 50 mM. The stability was excellent in all instances.

### Composition Example 1

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide; and the aqueous solutions of the compositions shown in Table 5 were prepared. The aqueous solutions of the compositions shown in Table 5 were subjected to aseptic filtration, and 5.21 mL of formulation solution example 1 and 20.66 ml of the formulation solution example 2 were filled into sterilized vials, respectively. After being frozen to a temperature of −40° C. or lower, the vials were depressurized to vacuum, and the water was removed by setting the shelf temperature to −10° C. Thereafter, the residual water was removed by setting the shelf temperature to 30° C., thus obtaining aseptic freeze-dried compositions having the compositions shown in Table 6. After storage at 40° C./75% RH (relative humidity) for 6 months, or at 25° C./60% RH for 36 months, the stability of compound A in the freeze-dried composition of formulation example 1 was analyzed. According to the results of the HPLC area normalization method, the amount of tolvaptan produced was below the detection limit; compound A was extremely stable, even after long-term storage. Water for injection (5 mL) was added to formulation example 1, and 20 mL of water for injection was added to formulation example 2 to achieve reconstitution. Thus, formulation solution example 1 and formulation solution example 2 shown in Table 5 were obtained in which insoluble foreign matter and insoluble particulate matter were within the range specified in the Japanese Pharmacopoeia, even after long-term storage.

TABLE 5
Formulation example of aqueous solution composition of
compound A (mg/mL)

| | Formulation | Formulation |
|---|---|---|
| | (Solution) | (Solution) |
| | Example 1 | Example 2 |
| Compound A | 5.00 | 25.52 |
| Sucrose | 48.00 | 20.00 |
| Sodium hydrogen phosphate hydrate | 18.00 | 18.00 |
| Sodium dihydrogen phosphate | 0.30 | 0.30 |

| | |
|---|---|
| Sodium hydroxide | q.s. to pH 8.5 |
| Water for injection | q.s. to 1.00 mL |

TABLE 6
Formulation example of freeze-dried composition of
compound A (mg/vial)

| | Formulation | Formulation |
|---|---|---|
| | Example 1 | Example 2 |
| Compound A | 26.05 | 527.24 |
| Sucrose | 250.08 | 413.20 |
| Sodium hydrogen phosphate hydrate | 93.78 | 371.88 |
| Sodium dihydrogen phosphate | 1.563 | 6.20 |

| | |
|---|---|
| Sodium hydroxide | q.s. |

### Composition Example 2

The following tables show composition examples of formulations comprising compound A. Compound A, purified white sugar (sucrose), sodium hydrogen phosphate hydrate, and sodium dihydrogen phosphate were dissolved in water for injection, the pH was adjusted to 8.5 with sodium hydroxide, and the aqueous solutions of the compositions shown in Table 7 were prepared. After aseptic filtration, 2.63 mL to 2.64 mL of the aqueous solutions of the compositions shown in Table 7 were filled into sterilized vials. After being frozen to a temperature of −40° C. or lower, the vials were depressurized to vacuum, and the water was removed by setting the shelf temperature to −20° C. Thereafter, the residual water was removed by setting the shelf temperature to 30° C., thus obtaining aseptic freeze-dried compositions having the compositions shown in Table 8. After storage at 40° C./75% RH for 6 months, or at 25° C./60% RE for 18 months, the stability of compound A in the freeze-dried compositions of formulation examples 3, 4, 5, and 6 of Table 5 was analyzed. The amounts of tolvaptan produced were all below the detection limit; compound A was extremely stable, even after long-term storage. Water for injection (2.5 mL) was added to the freeze-dried compositions shown in Table 7. Then, formulation solution examples 3, 4, 5, and 6 as shown in Table 7 were obtained in which insoluble foreign matter and insoluble particulate matter were within the range specified in the Japanese Pharmacopoeia, even after long-term storage.

TABLE 7
Formulation example of aqueous solution composition of
compound A (mg/mL)

| | Formulation | Formulation | Formulation | Formulation |
|---|---|---|---|---|
| | (Solution) | (Solution) | (Solution) | (Solution) |
| | Example 3 | Example 4 | Example 5 | Example 6 |
| Compound A | 1.00 | 2.00 | 4.00 | 8.00 |
| Sucrose | 70.10 | 68.40 | 65.10 | 58.50 |
| Sodium hydrogen | 9.0 | 9.0 | 9.0 | 9.0 |
| phosphate hydrate | | | | |
| Sodium dihydro- | 0.15 | 0.15 | 0.15 | 0.15 |
| gen phosphate | | | | |

| | |
|---|---|
| Sodium hydroxide | q.s. to pH 8.5 |
| Water for injection | q.s. to 1.00 mL |

TABLE 8
Formulation example of freeze-dried composition of compound A (mg/vial)

| | Formulation | Formulation | Formulation | Formulation |
|---|---|---|---|---|
| | Example 3 | Example 4 | Example 5 | Example 6 |
| Compound A | 2.64 | 5.28 | 10.56 | 21.04 |
| Sucrose | 185.06 | 180.58 | 171.86 | 153.86 |
| Sodium hydrogen | 23.76 | 23.76 | 23.76 | 23.67 |
| phosphate hydrate | | | | |
| Sodium dihydro- | 0.40 | 0.40 | 0.40 | 0.39 |
| gen phosphate | | | | |

| | |
|---|---|
| Sodium hydroxide | q s. |

### Composition Example 3

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide; and the aqueous solutions of the compositions shown in Table 9 were prepared. Further, after aseptic filtration, 2 mL of the aqueous solutions of the compositions shown in Table 9 were filled into vials. After being frozen to a temperature of −40° C. or lower, the vials were depressurized to vacuum, and the water was removed by setting the shelf temperature to −20° C. Thereafter, the residual water was removed by setting the shelf temperature to 30° C., thus obtaining freeze-dried compositions having the compositions shown in Table 10. After storage at 50° C. for 4 weeks, the stability of compound A in the freeze-dried compositions of formulation examples 7, 8, and 9 shown in Table 10 was analyzed. The amount of tolvaptan produced was 1.2% in formulation example 7 (comparative example), and below the detection limit in formulation examples 8 and 9. Fifty mL of saline was added to the freeze-dried compositions shown in Table 10 after storage at 50° C. for 4 weeks to achieve reconstitution. Although the insoluble particulate matter in formulation example 7 (comparative example) exceeded the amount specified in the Japanese Pharmacopoeia, the amount in formulation examples 8 and 9 were within the range specified in the Japanese Pharmacopoeia. Therefore, the effect of the addition of sucrose on formation of tolvaptan and insoluble particulate matter was confirmed here, as well.

TABLE 9
Formulation example of aqueous solution composition of
compound A (mg/mL)

| | Formulation | | |
|---|---|---|---|
| | (Solution) | | |
| | Example 7 | Formulation | Formulation |
| | (Comparative | (Solution) | (Solution) |
| | Example) | Example 8 | Example 9 |
| Compound A | 4.00 | 4.00 | 4.00 |
| Sucrose | 0 | 20.00 | 40.00 |
| Sodium hydrogen | 9.00 | 9.00 | 9.00 |
| phosphate hydrate | | | |
| Sodium dihydrogen | 0.15 | 0.15 | 0.15 |
| phosphate | | | |

| | |
|---|---|
| Sodium hydroxide | q.s. to pH 8.5 |
| Water for injection | q.s. to 1.00 mL |

TABLE 10
Formulation example of freeze-dried composition of
compound A (mg/vial)

| | Formulation | | |
|---|---|---|---|
| | (Solution) | | |
| | Example 7 | Formulation | Formulation |
| | (Comparative | (Solution) | (Solution) |
| | Example) | Example 8 | Example 9 |
| Compound A | 8.00 | 8.00 | 8.00 |
| Sucrose | 0 | 40.00 | 80.00 |
| Sodium hydrogen | 18.00 | 18.00 | 18.00 |
| phosphate hydrate | | | |
| Sodium dihydrogen | 0.3 | 0.3 | 0.3 |
| phosphate | | | |

| | |
|---|---|
| Sodium hydroxide | q.s. |

### Composition Example 4

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide; and the aqueous solutions of the compositions shown in Table 11 were prepared. After aseptic filtration, 2 mL of the aqueous solutions of the compositions shown in Table 11 were filled into vials. After being frozen to a temperature of −40° C. or lower, the vials were depressurized to vacuum, and the water was removed by setting the shelf temperature to −20° C. Thereafter, the residual water was removed by setting the shelf temperature to 30° C., thus obtaining freeze-dried compositions having the compositions shown in Table 12. After storage at 50° C. for 4 weeks, the stability of compound A in the freeze-dried compositions of formulation examples 10, 11, 12, and 13 shown in Table 12 was analyzed. The amount of tolvaptan produced was below the detection limit in all of the formulation examples. Fifty mL of saline was added to the freeze-dried compositions shown in Table 12 after storage at 50° C. for 4 weeks to achieve reconstitution, to thus obtain formulation solutions in which insoluble foreign matter and insoluble particulate matter were within the range specified in the Japanese Pharmacopoeia.

TABLE 11
Formulation example of aqueous solution composition of
compound A (mg/mL)

| | Formulation | Formulation | Formulation | Formulation |
|---|---|---|---|---|
| | (Solution) | (Solution) | (Solution) | (Solution) |
| | Example 10 | Example 11 | Example 12 | Example 13 |
| Compound A | 2.00 | 2.00 | 2.00 | 2.00 |
| Sucrose | 10.00 | 20.00 | 30.00 | 40.00 |
| Sodium hydrogen | 9.00 | 9.0 | 9.0 | 9.0 |
| phosphate hydrate | | | | |
| Sodium dihydro- | 0.15 | 0.15 | 0.15 | 0.15 |
| gen phosphate | | | | |

| | |
|---|---|
| Sodium hydroxide | q.s. to pH 8.5 |
| Water for injection | q.s. to 1.00 mL |

TABLE 12
Formulation example of freeze-dried composition of
compound A (mg/vial)

| | Formulation | Formulation | Formulation | Formulation |
|---|---|---|---|---|
| | Example 10 | Example 11 | Example 12 | Example 13 |
| Compound A | 4.00 | 4.00 | 4.00 | 4.00 |
| Sucrose | 20.00 | 40.00 | 60.00 | 80.00 |
| Sodium hydrogen | 18.00 | 18.00 | 18.00 | 18.00 |
| phosphate hydrate | | | | |
| Sodium dihydro- | 0.3 | 0.3 | 0.3 | 0.3 |
| gen phosphate | | | | |

| | |
|---|---|
| Sodium hydroxide | q s. |

### Composition Example 5

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide; and the aqueous solutions of the compositions shown in Table 13 were prepared. After aseptic filtration, 2 of the aqueous solutions of the compositions shown in Table 13 were filled into vials. After being frozen to a temperature of −40° C. or lower, the vials were depressurized to vacuum, and the water was removed by setting the shelf temperature to −20° C. Thereafter, the residual water was removed by setting the shelf temperature to 30° C., thus obtaining freeze-dried compositions having the compositions shown in Table 14. After storage at 50° C. for 4 weeks, the stability of compound A in the freeze-dried compositions of formulation examples 14, 15, 16, and 17 shown in Table 14 was analyzed. The amount of tolvaptan produced was below the detection limit in all of the formulation examples. 50 mL of saline was added to the freeze-dried compositions shown in Table 14 after storage at 50° C. for 4 weeks to achieve reconstitution, to thus obtain formulation solutions in which insoluble foreign matter and insoluble particulate matter were within the range specified in the Japanese Pharmacopoeia.

TABLE 13
Formulation example of aqueous solution composition of
compound A (mg/mL)

| | Formulation | Formulation | Formulation | Formulation |
|---|---|---|---|---|
| | (Solution) | (Solution) | (Solution) | (Solution) |
| | Example 14 | Example 15 | Example 16 | Example 17 |
| Compound A | 8.00 | 8.00 | 8.00 | 8.00 |
| Sucrose | 10.00 | 20.00 | 30.00 | 40.00 |
| Sodium hydrogen | 9.00 | 9.0 | 9.0 | 9.0 |
| phosphate hydrate | | | | |
| Sodium dihydro- | 0.15 | 0.15 | 0.15 | 0.15 |
| gen phosphate | | | | |

| | |
|---|---|
| Sodium hydroxide | q.s. to pH 8.5 |
| Water for injection | q.s. to 1.00 mL |

TABLE 14
Formulation example of freeze-dried composition of
compound A (mg/vial)

| | Formulation | Formulation | Formulation | Formulation |
|---|---|---|---|---|
| | Example 14 | Example 15 | Example 16 | Example 17 |
| Compound A | 16.00 | 16.00 | 16.00 | 16.00 |
| Sucrose | 20.00 | 40.00 | 60.00 | 80.00 |
| Sodium hydrogen | 18.00 | 18.00 | 18.00 | 18.00 |
| phosphate hydrate | | | | |
| Sodium dihydro- | 0.3 | 0.3 | 0.3 | 0.3 |
| gen phosphate | | | | |

| | |
|---|---|
| Sodium hydroxide | q.s. |

### Composition Example 6

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide; and the aqueous solutions of the compositions shown in Table 15 were prepared. After aseptic filtration, 2.04 mL of the aqueous solutions of the compositions shown in Table 15 were filled in vials. After being frozen to a temperature of −40° C. or lower, the vials were depressurized to vacuum, and the water was removed by setting the shelf temperature to −20° C. Thereafter, the residual water was removed by setting the shelf temperature to 30° C., thus obtaining freeze-dried compositions having the compositions shown in Table 16. The freeze-dried compositions of Table 16 were dissolved in 50 mL of physiological saline or glucose injection solution, to thus prepare injection solutions of compound A for infusion.

TABLE 15
Formulation example of aqueous solution composition of
compound A (mg/mL)

| | Formulation | Formulation |
|---|---|---|
| | (Solution) | (Solution) |
| | Example 8 | Example 15 |
| Compound A | 4.00 | 8.00 |
| Sucrose | 20.00 | 20.00 |
| Sodium hydrogen phosphate hydrate | 9.00 | 9.00 |
| Sodium dihydrogen phosphate | 0.15 | 0.15 |

| | |
|---|---|
| Sodium hydroxide | q.s. to pH 8.5 |
| Water for injection | q.s. to 1.00 mL |

TABLE 16
Formulation example of freeze-dried composition of
compound A (mg/vial)

| | Formulation | Formulation |
|---|---|---|
| | Example 18 | Example 19 |
| Compound A | 8.16 | 16.32 |
| Sucrose | 40.80 | 40.80 |
| Sodium hydrogen phosphate hydrate | 18.36 | 18.36 |
| Sodium dihydrogen phosphate | 0.306 | 0.306 |

| | |
|---|---|
| Sodium hydroxide | q.s. |

### Composition Example 7

The following tables show composition examples of formulations comprising compound A. Compound A, purified white sugar (sucrose), sodium hydrogen phosphate hydrate, and sodium dihydrogen phosphate were dissolved in water for injection; the pH was adjusted to 8.5 with sodium hydroxide or phosphoric acid; and the aqueous solutions of the compositions shown in Table 17 were prepared. After aseptic filtration, 2.14 mL of the aqueous solutions of the compositions shown in Table 17 were filled into vials. After being frozen to a temperature of −40° C. or lower, the vials were depressurized to vacuum, and the water was removed by setting the shelf temperature to −10° C. Thereafter, the residual water was removed by setting the shelf temperature to 40° C., thus obtaining freeze-dried compositions having the compositions shown in Table 18. The freeze-dried compositions of Table 18 were dissolved in 50 mL of physiological saline or glucose injection solution, to thus prepare injection solutions of compound A for infusion.

TABLE 17
Formulation example of aqueous solution composition of
compound A (mg/mL)

| | Formulation | Formulation |
|---|---|---|
| | (Solution) | (Solution) |
| | Example 8 | Example 15 |
| Compound A | 4.00 | 8.00 |
| Sucrose | 20.00 | 20.00 |
| Sodium hydrogen phosphate hydrate | 9.00 | 9.00 |
| Sodium dihydrogen phosphate | 0.15 | 0.15 |

| | |
|---|---|
| Sodium hydroxide | q.s. to pH 8.5 |
| Phosphoric acid | q.s. to pH 8.5 |
| Water for injection | q.s. to 1.00 mL |

TABLE 18
Formulation example of freeze-dried composition of
compound A (mg/vial)

| | Formulation | Formulation |
|---|---|---|
| | Example 20 | Example 21 |
| Compound A | 8.56 | 17.12 |
| Sucrose | 42.80 | 42.80 |
| Sodium hydrogen phosphate hydrate | 19.26 | 19.26 |
| Sodium dihydrogen phosphate | 0.321 | 0.321 |

| | |
|---|---|
| Sodium hydroxide | q.s. |
| Phosphoric acid | q.s. |

1. A freeze-dried composition comprising a compound represented by Formula (1): 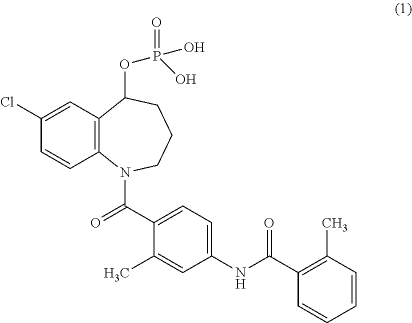 [SMILES: Cc1ccccc1C(=O)Nc1ccc(C(=O)N2CCCC(OP(=O)(O)O)c3cc(Cl)ccc32)c(C)c1]
or a metal salt thereof and a disaccharide.

2. The freeze-dried composition according to claim 1, wherein the metal salt is a disodium salt.

3. The freeze-dried composition according to claim 1, wherein the disaccharide is at least one member selected from the group consisting of sucrose, maltose, lactose, and trehalose.

4. The freeze-dried composition according to claim 1, wherein the disaccharide is present in an amount of 0.5 to 70 parts by mass per part by mass of the compound represented by Formula (1) or a metal salt thereof.

5. The freeze-dried composition according to claim 1, wherein the total amount of the compound represented by Formula (1) or a metal salt thereof and the disaccharide is 65% by mass or more of the entire composition.

6. The freeze-dried composition according to claim 1, further comprising a buffering agent.

7. The freeze-dried composition according to claim 6, wherein the buffering agent is a phosphate buffering agent.

8. The freeze-dried composition according to claim 1, which is for use by transvascular administration after dissolving in water so as to constitute an aqueous solution composition having a pH of 7 to 9.

9. An aqueous solution composition comprising a compound represented by Formula (1): 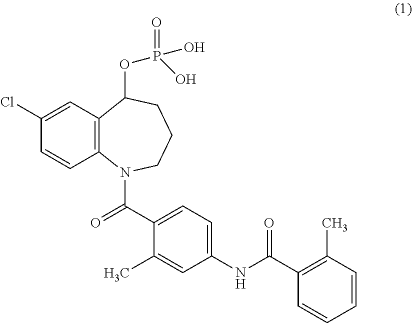 [SMILES: Cc1ccccc1C(=O)Nc1ccc(C(=O)N2CCCC(OP(=O)(O)O)c3cc(Cl)ccc32)c(C)c1]
or a metal salt thereof and a disaccharide, and having a pH of 7 to 9.

10. The aqueous solution composition according to claim 9, wherein the metal salt is a disodium salt.

11. The aqueous solution composition according to claim 9, wherein the disaccharide is at least one member selected from the group consisting of sucrose, maltose, lactose, and trehalose.

12. The aqueous solution composition according to claim 9, wherein the disaccharide is present in an amount of 0.5 to 70 parts by mass per part by mass of the compound represented by Formula (1) or a metal salt thereof.

13. The aqueous solution composition according to claim 9, wherein the disaccharide is present at a concentration of 1 to 8 w/v %.

14. The aqueous solution composition according to claim 9, further comprising a buffering agent.

15. The aqueous solution composition according to claim 14, wherein the buffering agent is a phosphate buffering agent.

16. The aqueous solution composition according to claim 9, which is for transvascular administration.

17. The aqueous solution composition according to claim 9, for use in the preparation of a freeze-dried composition comprising a compound represented by Formula (1): 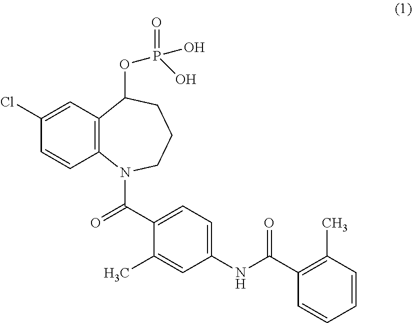 [SMILES: Cc1ccccc1C(=O)Nc1ccc(C(=O)N2CCCC(OP(=O)(O)O)c3cc(Cl)ccc32)c(C)c1]
or a metal salt thereof and a disaccharide.

18. The freeze-dried composition according to claim 1, for use in the preparation of an aqueous solution composition comprising a compound represented by Formula (1): 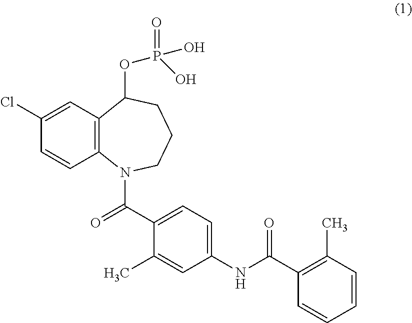 [SMILES: Cc1ccccc1C(=O)Nc1ccc(C(=O)N2CCCC(OP(=O)(O)O)c3cc(Cl)ccc32)c(C)c1]
or a metal salt thereof and a disaccharide, and having a pH of 7 to 9.

19. The composition according to claim 1, which is sterilized.

20. The composition according to claim 1, which is a pharmaceutical composition.
